(19) United States
(12) Patent Application Publication
(10) Pub. No.: US 2018/0027766 A1
(43) Pub. Date: Feb. 1, 2018

(54) ANIMAL IDENTIFICATION THROUGH ULTRAVIOLET LIGHT-REACTIVE TATTOO

(71) Applicant: Justin T. Doubet, Parker, CO (US)

(72) Inventor: Justin T. Doubet, Parker, CO (US)

(21) Appl. No.: 15/225,376

(22) Filed: Aug. 1, 2016

(51) Int. Cl. A01K 11/00; A61D 99/00; A61M 37/00

(52) U.S. Cl. CPC A01K 11/005; A61M 37/0076; A61D 99/00

(57) ABSTRACT

Improved animal identification using light-reactive ink. An embodiment comprises: causing a pattern of holes to be formed in a portion of a body of an animal; and introducing light-reactive ink into the holes, thereby forming an identifying mark in the portion as a tattoo, the ink being reactive to ultraviolet light such that the identifying mark becomes viewable in response to introduction of ultraviolet light. Introducing an ultraviolet light source in a vicinity of the portion of the body causes the identifying mark to luminesce and thus be visible by a human. The light-reactive ink may contain fluorescent or phosphorescent dye. The portion of the body may be an inner ear of the animal. A color of the ink may substantially match a color of the inner ear of the animal and upon introduction of the ultraviolet light, may have a color different from the color of the inner ear.

### BACKGROUND

The present invention relates to an improved approach for animal identification.

Animals need identification for various purposes, including theft prevention and confirmation of identity for verification of bloodline.

### BRIEF SUMMARY

The present invention is directed to an improved approach for animal identification. In one aspect, this comprises causing a pattern of holes to be formed in a portion of a body of an animal; and introducing light-reactive ink into the holes, thereby forming an identifying mark in the portion of the body, the ink being reactive to ultraviolet light such that the identifying mark becomes viewable in response to introduction of ultraviolet light. Introducing an ultraviolet light source in a vicinity of the portion of the body thereby causes the identifying mark to luminesce and thus be visible by a human. In one approach, the light-reactive ink contains fluorescent dye; in another approach, the light-reactive ink contains phosphorescent dye. The portion of the body may be an inner ear of the animal. A color of the ink may substantially match a color of the inner ear of the animal and upon introduction of the ultraviolet light, have a color different from the color of the inner ear. Introduction of the light-reactive ink preferably causes the ink to settle permanently into a dermis layer of the inner ear. The light-reactive ink may be introduced into the holes by application in a liquid form or in another approach, in a paste form. Causing the pattern of holes to be formed preferably comprises: placing a clamping device over the body portion, the clamping device containing a plurality of needles arranged into the pattern; and applying pressure to the clamping device so as to cause the needles to form the holes. In another aspect, an embodiment of the present invention comprises a clamping device adapted for holding at least one needle insert, each needle insert comprising a plurality of needles arranged into a shape, the clamping device being operable to cause the needles to create a corresponding plurality of holes in a portion of a body of an animal upon application of pressure thereto; light-reactive ink for introduction into the holes to thereby form an identifying mark in the portion of the body, the ink being reactive to ultraviolet light such that the identifying mark becomes viewable in response to introduction of an ultraviolet light source; and optionally, the ultraviolet light source. The identifying mark is preferably not viewable in absence of the ultraviolet light source. Introducing the ultraviolet light source causes the identifying mark to luminesce. Introduction of the light-reactive ink preferably causes the ink to settle permanently, as a tattoo, into a dermis layer of the body portion.

Various embodiments of these and other aspects of the present invention may be provided in view of the present disclosure. It should be noted that the foregoing is a summary and thus contains, by necessity, simplifications, generalizations, and omissions of detail; consequently, those skilled in the art will appreciate that the summary is illustrative only and is not intended to be in any way limiting. Other aspects, inventive features, and advantages of the present invention, as defined by the appended claims, will become apparent in the non-limiting detailed description set forth below.

### BRIEF DESCRIPTION OF THE SEVERAL VIEWS OF THE DRAWINGS

The present invention will be described with reference to the following drawings, in which like reference numbers denote the same element throughout.

FIG. 1 depicts a clamping device usable for tattooing an animal;

FIG. 2 illustrates inserts that may be placed into the clamping device illustrated in FIG. 1, where these inserts comprise needles pre-formed into alphabetic and/or numeric shapes; and

FIG. 3 illustrates how an animal identifying tattoo created according to an embodiment of the present invention may be viewed upon introduction of ultraviolet light.

### DETAILED DESCRIPTION

As noted earlier, animals need identification for various purposes, including theft prevention and confirmation of identity for verification of bloodline. Proper animal identification may be of concern whether the animal is a family pet, part of a livestock operation, is the subject of research, and so forth.

Traditional means for marking animals for identification have varied, and different techniques have been commonly used for different types of animals. Branding an animal's skin or hide, for example, has been commonly used for cattle and horses. Attaching a plastic or metal tag to an animal's ear is common with cattle and hogs, although these tags can be removed either by accident (e.g., if the animal's ear comes in contact with a surface that catches or snags the tag) or intentionally (e.g., by a thief who simply cuts the tag loose from a stolen animal). Implantable devices such as radio-frequency identifier (“RFID”) tags have been used more recently, although thieves have been known to remove or destroy such implantable devices.

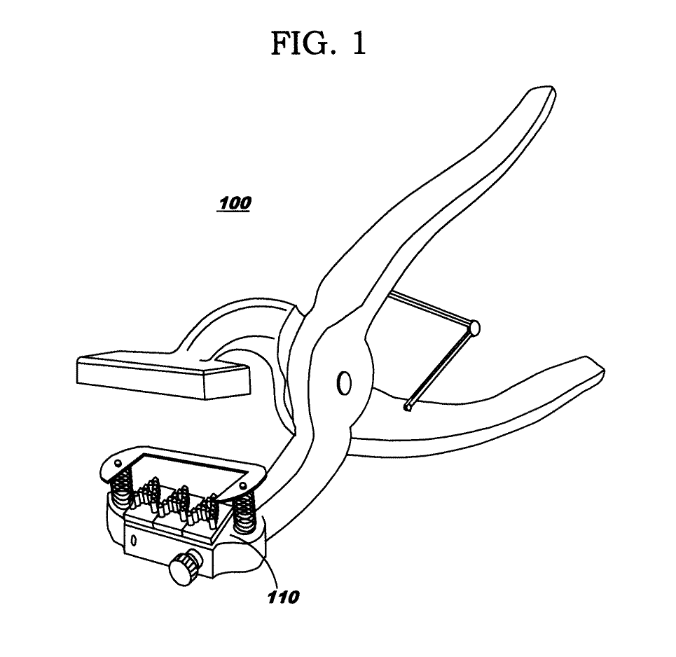

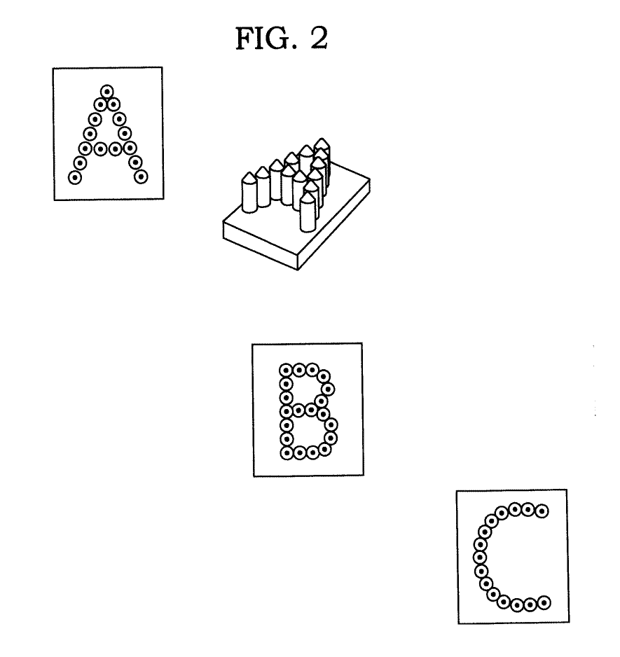

In view of drawbacks of removable devices and techniques, animal owners often desire to mark their animals in a permanent manner. Identifying animals by tattooing is one such approach for permanent identification. Commonly, so-called “tattoo pliers” are used to place an identifying tattoo into an animal's ear. An example of tattoo pliers is depicted at 100 in FIG. 1, and comprises a clamping-style device having a tray or holder 110 on which needles pre-formed into alphabetic and/or numeric shapes are placed. To mark an animal with identifier “A123”, for example, an insert for the letter “A” is placed into the tray or holder of the clamping device, along with inserts for each of the numbers “1”, “2”, and “3”. FIG. 2 illustrates examples of such inserts, having needles that are formed into particular shapes.

The animal to be tattooed is commonly placed in a restraint during the tattooing process. An interior area of the animal's ear may then be cleaned to remove dirt or other substances. Any hair on the interior area may be shaved away, if needed. The clamping device is placed over the interior area of the ear, and pressure is applied so as to cause the needle inserts to make corresponding holes in the animal's ear. Tattoo ink is introduced into these holes. The ink may be introduced to the ear after the holes are made, for example by rolling a ball affixed to a liquid ink dispenser over the area that now contains the holes or by applying ink in a paste form over the area containing the holes, causing the ink to enter into the holes. Or, the ink may be introduced to the surface of the animal's ear prior to applying the clamping device, so that the needles on each insert will drive ink into the holes at time of creation. In either approach, the tattoo ink enters the dermis layer in the shape or pattern created by the holes, where it will remain in a generally permanent manner. Any excess ink (i.e., ink that does not enter the holes) on the inner ear surface may optionally be removed afterward, such as by wiping with a cloth.

After tattooing an animal in this manner, the tattoo may be read by inspecting the animal's ear, and the identifying mark can be seen by the ink that remains in the holes created by the needles. This known approach to tattooing works well in many scenarios. However, several problems may arise. Animal tattoo inks are most often black in color, although green ink and white ink are known to be used as well. If an animal has skin or hide of a black, brown, or other dark color, a tattoo created using black ink will generally not be visible (i.e., unless the animal has light-colored skin inside its ear). Green ink will also not generally be visible on such animals, and thus white ink must be used. Tattoos using white ink can still be difficult to see, however. And, even for those animals whose inner ear surface is light in color, it is generally necessary to be in very close proximity to the animal's ear for a human being to read a tattoo created using black or green ink—and similarly for a tattoo created using white ink for an animal having a dark-colored inner ear surface. For animals that are not domesticated, confining or restraining the animal in order to manually inspect its ear is sometimes not feasible, particularly when a large number of animals need to be identified. Accordingly, known techniques for animal identification using tattoos are somewhat inconvenient. They may also be error-prone, as there is some likelihood of misreading a tattoo due to insufficient contrast between the tattoo ink and the skin and/or due to inability to closely view the tattoo.

The present invention is directed to improved animal identification. In an embodiment, an ink that is reactive to non-visible light is used in the tattooing process, and is preferably introduced into holes in a body portion of an animal, the holes created by needles in a clamping device that is applied to the body portion, where introduction of the ink into the holes forms an identifying mark on the body portion of the animal. Preferably, the reactive ink contains a fluorescent dye. Fluorescent dye can be made to luminesce in the presence of ultraviolet light. The reactive ink may alternatively contain a phosphorescent dye, which will also luminesce in the presence of ultraviolet light. As is known to those of skill in the art, visible light has longer wavelengths than ultraviolet light, and thus ultraviolet light is not visible to the human eye. An embodiment of the present invention introduces ultraviolet light in the vicinity of the tattooed area with a device that emits ultraviolet light but blocks (at least most) visible light, for example by using a bulb formed from a special type of glass that blocks all visible light above a particular wavelength (such as approximately 400 nanometers). One such glass is a nickel-oxide doped glass known as “Wood's glass”. Such bulbs are commonly referred to as “blacklight” bulbs or a “Wood's light”.

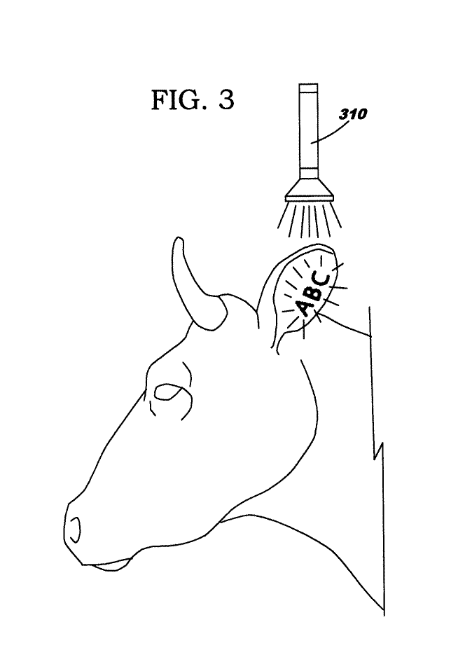

Introduction of the ultraviolet light in the vicinity of the tattooed area (whether it is an animal's ear or other location on the animal) will react with the fluorescent ink of the tattoo, causing the tattoo to luminesce, or glow, and thus be viewable to a human—appearing, that is, as a series of “glowing” alphabetic and/or numeric shapes. See FIG. 3, which illustrates a tattoo comprising the letters “ABC” in the ear of a cow, which appears to glow upon introduction of ultraviolet light source 310. (Note that the letters and/or numbers in an actual animal tattoo are generally smaller than the illustration in FIG. 3, as the letters in this example have been enlarged for readability.) Similarly, phosphorescent ink will also luminesce as a reaction to introduction of ultraviolet light. The luminescence occurs as phosphors in the dye convert energy from the ultraviolet light into visible light. An animal's identifying tattoo can thus be observed without regard to the color of the animal's skin or hide, and without a requirement to be in very close proximity to the actual tattooed area. Animal identification is thus more convenient and less error-prone as compared to known techniques.

Embodiments of the present invention have been discussed primarily with reference to a tattoo placed inside an animal's ear. This is by way of illustration and not of limitation, and animal identifying tattoos placed in other bodily locations are also deemed to be within the scope of the present invention.

According to the above-described improvements for improved animal identification, an embodiment of the present invention comprises a method of marking animals by tattooing using light-reactive ink that is viewable in response to introduction of ultraviolet light. Another embodiment comprises a tattooing system that includes reactive ink, the ink being reactive to ultraviolet light.

While embodiments of the present invention have been described, additional variations and modifications in those embodiments may occur to those skilled in the art once they learn of the basic inventive concepts. Therefore, it is intended that the appended claims shall be construed to include the described embodiments and all such variations and modifications as fall within the spirit and scope of the invention.

1. An improved animal identification method, comprising:
causing a pattern of holes to be formed in a portion of a body of an animal; and
introducing light-reactive ink into the holes, thereby forming an identifying mark in the portion of the body, the ink being reactive to ultraviolet light such that the identifying mark becomes viewable in response to introduction of ultraviolet light.

2. The method according to claim 1, further comprising introducing an ultraviolet light source in a vicinity of the portion of the body, thereby causing the identifying mark to luminesce and thus be visible by a human.

3. The method according to claim 1, wherein the light-reactive ink contains fluorescent dye.

4. The method according to claim 1, wherein the light-reactive ink contains phosphorescent dye.

5. The method according to claim 1, wherein the portion of the body is an inner ear of the animal.

6. The method according to claim 5, wherein a color of the ink substantially matches a color of the inner ear of the animal and upon introduction of the ultraviolet light, has a color different from the color of the inner ear.

7. The method according to claim 1, wherein the portion of the body is an inner ear of the animal and the introduction of the light-reactive ink causes the ink to settle permanently, as a tattoo, into a dermis layer of the inner ear.

8. The method according to claim 1, wherein the light-reactive ink is introduced into the holes by application in a liquid form.

9. The method according to claim 1, wherein the light-reactive ink is introduced into the holes by application in a paste form.

10. The method according to claim 1, wherein causing the pattern of holes to be formed comprises:
placing a clamping device over the body portion, the clamping device containing a plurality of needles arranged into the pattern; and
applying pressure to the clamping device so as to cause the needles to form the holes.

11. A system for animal identification, comprising:
a clamping device adapted for holding at least one needle insert, each needle insert comprising a plurality of needles arranged into a shape, the clamping device being operable to cause the needles to create a corresponding plurality of holes in a portion of a body of an animal upon application of pressure thereto; and
light-reactive ink for introduction into the holes to thereby form an identifying mark in the portion of the body, the ink being reactive to ultraviolet light such that the identifying mark becomes viewable in response to introduction of an ultraviolet light source.

12. The system according to claim 11, wherein the identifying mark is not viewable in absence of the ultraviolet light source.

13. The system according to claim 11, wherein introducing the ultraviolet light source causes the identifying mark to luminesce.

14. The system according to claim 11, wherein the system further comprises the ultraviolet light source.

15. The system according to claim 11, wherein the light-reactive ink contains fluorescent dye.

16. The system according to claim 11, wherein the light-reactive ink contains phosphorescent dye.

17. The system according to claim 11, wherein the portion of the body is an inner ear of the animal.

18. The system according to claim 17, wherein a color of the ink substantially matches a color of the inner ear of the animal and upon introduction of the ultraviolet light, has a color different from the color of the inner ear.

19. The system according to claim 11, wherein the portion of the body is an inner ear of the animal and the introduction of the light-reactive ink causes the ink to settle permanently, as a tattoo, into a dermis layer of the inner ear.

20. The system according to claim 11, wherein the light-reactive ink is introduced into the holes by application in a liquid form.
